(19) United States
(12) Patent Application Publication
(10) Pub. No.: US 2026/0325493 A1
(43) Pub. Date: Oct. 22, 2026

(54) AQUEOUS PHARMACEUTICAL COMPOSITION OF NITROFURANTOIN

(71) Applicant: Casper Pharma LLC, East Brunswick, NJ (US)

(72) Inventors: Vimal KAVURU, East Brunswick, NJ (US); Venkata Srikanth MEKA, Pedavegi (IN)

(21) Appl. No.: 19/681,959

(22) Filed: May 19, 2026

Related U.S. Application Data
(63) Continuation of application No. 17/963,530, filed on Oct. 11, 2022, now abandoned.

(51) Int. Cl. A61K 31/4178; A61K 9/00; A61K 47/02; A61K 47/10; A61K 47/12; A61K 47/14; A61K 47/26; A61K 47/38

(52) U.S. Cl. CPC A61K 31/4178; A61K 9/0053; A61K 47/02; A61K 47/10; A61K 47/12; A61K 47/14; A61K 47/26; A61K 47/38

(57) ABSTRACT

Provided herein are aqueous pharmaceutical compositions of nitrofurantoin for oral administration containing nitrofurantoin as the active pharmaceutical ingredient and one more excipient selected from suspending agents, binders, preservatives, vehicles, sweetening agents, buffering agents and taste masking agents.

### FIELD OF THE INVENTION

The invention relates to pharmaceutical liquid suspension formulations suitable for oral administration. In particular, the invention relates oral pharmaceutical suspensions containing nitrofurantoin.

### DESCRIPTION OF RELATED ART

Nitrofurantoin is a synthetic antibacterial agent specifically indicated for the treatment of urinary tract infections. The chemical name of nitrofurantoin is 1-[[[5-nitro-2-furanyl]methylene]amino]-2,4-imidazolidinedione. The chemical structure is the following:

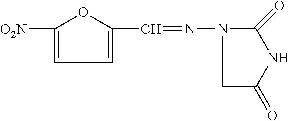 [SMILES: O=C1CN(N=Cc2ccc([N+](=O)[O-])o2)C(=O)N1]

Nitrofurantoin was first approved by the US FDA in 1954 and has merged as first-line UTI therapy due to the emergence of resistance to other antibiotics such as co-trimoxazole, trimethoprim, the fluoroquinolones and amoxicillin. Due to the poor pK profile, nitrofurantoin is administered in different dosage forms, doses, crystal forms, to maximise the bioavailability of nitrofurantoin and to minimize the toxicity. See, for example, Rixt A Wijma et al, “Review of the pharmacokinetic properties of nitrofurantoin and nitroxoline”, Journal of Antimicrobial Chemotherapy, Volume 73, Issue 11, November 2018, (2916-2926).

Nitrofurantoin is commercially available in multiple dosage forms and strengths, as tablets containing 50 mg or 100 mg of nitrofurantoin, capsules containing 25 mg, 50 or 100 mg of nitrofurantoin and as an oral suspension containing 25 mg/5 ml of nitrofurantoin. For example, U.S. Pat. Nos. 4,122,157, 4,370,313, EP Publication No. 250,023, and EP Publication No. 250,038, describe solid oral dosage forms of nitrofurantoin and N. Shah et al., “Effect of Polymers on Dissolution from Drug Suspensions”, 65 J. Pharmaceutical Sciences 1618 (1976) and U.S. Pat. No. 5,178,880 describe oral liquid dosage forms of nitrofurantoin.

The current approved oral suspension dosage forms of nitrofurantoin are found to have elemental impurities beyond the ICH acceptable limits. Therefore, there exists a need for nitrofurantoin oral suspension dosage forms that contain lower levels of elemental impurities which are below the acceptance limits.

### SUMMARY OF THE INVENTION

The invention disclosed herein provides an aqueous pharmaceutical suspension for oral administration consisting nitrofurantoin or its pharmaceutically salt or polymorph, at least one suspending agent, at least one binder, at least one preservative, at least one vehicle, at least one sweetening agent, at least one buffering agent and at least one taste masking flavour and wherein the amount of elemental impurities (lead content) in the said aqueous pharmaceutical suspension is less than 5 ppm.

The aqueous pharmaceutical suspension of this invention wherein the nitrofurantoin is selected from the group consisting of nitrofurantoin citrate, nitrofurantoin sodium and nitrofurantoin monohydrate.

The aqueous pharmaceutical suspension of this invention consists of nitrofurantoin or its pharmaceutically acceptable salt or polymorph in an amount of about 5 mg/ml to 10 mg/ml, at least one suspending agent in an amount of about 0.5 to 5%, at least one binder in an amount of about 3.0 to 6.0%, at least one preservative in an amount of about 0.015 to 0.20%, at least one vehicle in an amount of about 50 to 80%, at least one sweetening agent in an amount of about 50 to 80%, at least one buffering agent and at least one taste masking flavour.

The aqueous pharmaceutical suspension of this invention consists of nitrofurantoin or its pharmaceutically acceptable salt or polymorph, magnesium aluminium silicate, carboxymethylcellulose sodium, methyl paraben, propyl paraben, glycerin, sorbitol, sodium citrate dihydrate, taste masking agents and purified water and wherein the amount of lead content in the said aqueous pharmaceutical suspension is less than 5 ppm.

### DETAILED DESCRIPTION OF THE INVENTION

The invention as disclosed herein encompasses aqueous pharmaceutical suspension for oral administration consisting nitrofurantoin or its pharmaceutically salt or polymorph, having less than 5 ppm of lead content.

As disclosed herein, the aqueous pharmaceutical suspension for oral administration consists of therapeutically acceptable amount of nitrofurantoin or its pharmaceutically salt or polymorph, at least one suspending agent, at least one binder, at least one preservative, at least one vehicle, at least one sweetening agent, at least one buffering agent and at least one taste masking flavour.

Preferably, the aqueous pharmaceutical suspension of nitrofurantoin as disclosed herein consists of nitrofurantoin or its pharmaceutically acceptable salt or polymorph in an amount of about 5 mg/ml to 10 mg/ml, at least one suspending agent in an amount of about 0.5 to 5%, at least one binder in an amount of about 3.0 to 6.0%, at least one preservative in an amount of about 0.015 to 0.20%, at least one vehicle in an amount of about 50 to 80%, at least one sweetening agent in an amount of about 50 to 80%, at least one buffering agent and at least one taste masking flavor, wherein the pH of the said aqueous pharmaceutical suspension is from about 4.5 to 6.5, preferably 5.1 to 5.7, and the said nitrofurantoin active ingredient is selected from the group consisting of nitrofurantoin sodium, nitrofurantoin citrate and nitrofurantoin monohydrate.

The aqueous pharmaceutical suspension of this invention consists of 5 mg/ml of nitrofurantoin or its pharmaceutically acceptable salt or polymorph, magnesium aluminium silicate, carboxymethylcellulose sodium, methyl paraben, propyl paraben, glycerin, sorbital, sodium citrate dihydrate, taste masking agents and purified water and wherein the amount of lead content in the said aqueous pharmaceutical suspension is less than 5 ppm, preferably the lead content is less than 4 ppm, more preferably the lead content is less than 3.5 ppm.

The aqueous pharmaceutical suspension of this invention consists of 10 mg/ml of nitrofurantoin or its pharmaceutically acceptable salt or pols morph, magnesium aluminium silicate, carboxymethylcellulose sodium, methyl paraben, propyl paraben, glycerin, sorbital, sodium citrate dihydrate, taste masking agents and purified water and wherein the amount of lead content in the said aqueous pharmaceutical suspension is less than 5 ppm, preferably the lead content is less than 4 ppm, more preferably the lead content is less than 3.5 ppm.

The compositions of this invention employ a binding agent selected from the group comprising of Hypromellose, sodium carboxymethyl cellulose, Hydroxy propyl cellulose, xantham gum, carbomer, Carbopol, Polyvinyl Alcohol, Polyvinyl Pyrolidone iodine complex (PVC), and mixtures thereof.

The compositions of this invention employ a suspending agent selected from the group comprising of cellulose ethers (such as methylcellulose, hydroxyethylcellulose, and carboxymethylcellulose), alginates, carboxyvinylpolymers, xanthan gum, colloidal silicas montmorillonite clays and hydrophobically treated montmorillonite clays (such as magnesium aluminium silicate), and mixtures thereof. Preferred suspending agent include magnesium aluminium silicate or mixtures of cellulose ethers and magnesium aluminium silicate.

The compositions of this invention employ a buffering agent selected from the group comprising of Sulfamic acid/sulfamate, Formic acid/formate, Acetic acid/acetate, Dihydrogenphosphate/hydrogenphosphate, Ammonium ammonia, Bicarbonate/carbonate, Fumaric acid/hydrogen fumarate and Benzoic acid/benzoate, Sodium Citrate, Borate, Ammonium Carbonate, Calcium Carbonate, Citric Acid, Citric Acid, Trisodium Dihydrate, Citric Acid, Trisodium Dihydrate Proteomics Grade, Citric Acid, Ammonium Salt, Dibasic, UltraPure, Glycine, Ammonium Phosphate Dibasic, Potassium Phosphate Monobasic, Potassium Phosphate Tribasic, Sodium Phosphate Dibasic, Heptahydrate, and mixtures thereof.

The compositions of this invention employ a preserving agent selected from the group comprising of Methylparaben, Benzyl alcohol, Chlorobutanol, Ethanol, Phenylethyl alcohol, Phenoxyethanol, Propyl paraben, Ethyl paraben, Butyl paraben, Benzoic acid, Potassium benzoate, Sodium benzoate, and mixtures thereof.

The compositions of this invention employ a vehicle selected from the group comprising of glycerin. Sorbitol, Water. Oil, and mixtures thereof.

The compositions of this invention employ a sweetening agent selected from the group comprising of sorbitol, Sucrose Syrup, Glucose. Saccharin sodium. Sucralose, and mixtures thereof.

The pH of the aqueous pharmaceutical composition of nitrofurantoin as claimed herein may be adjusted by addition of a pharmaceutically-acceptable acid or base. Suitable acids include, for example, hydrochloric acid and carboxylic acids such as citric acid, tartaric acid and succinic acid. Suitable bases include, for example, the oxides and hydroxides of calcium, potassium, sodium and magnesium, alkaline quaternary compounds, alkaline amino acids, and mixtures thereof.

The invention will now be described with respect to the following specific examples.

### Example 1

The aqueous pharmaceutical composition of nitrofurantoin as disclosed herein has the following composition:

| S.No | Ingredients | Range |
|---|---|---|
| 1 | Nitrofurantoin monohydrate USP | 5 to 10 |
| | | mg/ml |
| 2 | Carboxymethylcellulose sodium | 3.0-6.0 |
| 3 | Magnesium aluminum silicate | 0.5-5% |
| 4 | Methyl paraben | 0.015-0.20 |
| 5 | Propyl paraben | 0.01-0.02 |
| 6 | Glycerin | 50-80% |
| 7 | Sorbitol solution | 50-80% |
| 8 | Sodium citrate dihydrate | 0.3-2.0% |
| 9 | Citric acid (anhydrous) | 0.3-2.0% |
| 10 | Primary taste modifier | 0.01-2.0% |
| 11 | Banana flavor | 0.01-2.0% |
| 12 | Rasberry flavor | 0.01-2.0% |

The aqueous pharmaceutical composition of this example consists of 50 mg/5 ml of nitrofurantoin and has a pH of 5.1 to 5.7.

### Example 2

According to one embodiment, the aqueous pharmaceutical composition of nitrofurantoin is as follows:

| S.No | Ingredients | mg/5 ml |
|---|---|---|
| 1 | Nitrofurantoin | 25.000 |
| 2 | Carboxymethylcellulose sodium | 100.000 |
| 3 | Magnesium aluminum silicate | 20.000 |
| 4 | Methyl paraben | 6.000 |
| 5 | Propyl paraben | 1.000 |
| 6 | Glycerin | 700.000 |
| 7 | Sorbitol solution | 700.000 |
| 8 | Sodium citrate dihydrate | 20.000 |
| 9 | Citric acid (anhydrous) | 6.800 |
| 10 | Primary taste modifier | 45.000 |
| 11 | Banana flavor | 0.250 |
| 12 | Rasberry flavor | 0.500 |
| 13 | Purified Water | q.s |

The aqueous pharmaceutical composition of this example consists of 25 mg/5 ml of nitrofurantoin and has a pH of 5.1 to 5.7.

### Example 3

According to one embodiment, the aqueous pharmaceutical composition of nitrofurantoin is as follows:

| S.No | Ingredients | mg/5 ml |
|---|---|---|
| 1 | Nitrofurantoin | 50.000 |
| 2 | Carboxymethylcellulose sodium | 60.000 |
| 3 | Magnesium aluminum silicate | 45.000 |
| 4 | Methyl paraben | 6.000 |
| 5 | Propyl paraben | 1.000 |
| 6 | Glycerin | 700.000 |
| 7 | Sorbitol solution | 700.000 |
| 8 | Sodium citrate dihydrate | 20.000 |
| 9 | Citric acid (anhydrous) | 6.800 |
| 10 | Primary taste modifier | 45.000 |
| 11 | Banana flavor | 0.250 |
| 12 | Rasberry flavor | 0.500 |
| 13 | Purified Water | q.s |

The aqueous is pharmaceutical composition of this example consists of 50 mg/5 ml of nitrofurantoin and has a pH of 5.1 to 5.7. The lead content in the abo e formulation is 3.07 PPM.

### Example 4

According to one embodiment, the aqueous pharmaceutical composition of nitrofurantoin is as follows:

| S.No | Ingredients | mg/5 mml |
|---|---|---|
| 1 | Nitrofurantoin monohy drate | 50.000 |
| 2 | Carboxymethylcellulose sodium | 70.000 |
| 3 | Magnesium aluminum silicate | 40.000 |
| 4 | Methyl paraben | 6.000 |
| 5 | Propyl paraben | 1.000 |
| 6 | Glycerin | 700.000 |
| 7 | Sorbitol solution | 700.000 |
| 8 | Sodium citrate dihydrate | 20.000 |
| 9 | Citric acid (anhydrous) | 6.800 |
| 10 | Primary taste modifier | 45.000 |
| 11 | Banana flavor | 0.250 |
| 12 | Rasberry flavor | 0.500 |
| 13 | Purified Water | q.s |

The aqueous pharmaceutical composition of the example consists of 50 mg/5 ml of nitrofurantoin and has a pH of 5.1 to 5.7. The lead content in the abo e formulation is 3.24 PPM.

### Example 5

According to one embodiment, the aqueous pharmaceutical composition of nitrofurantoin is as follows:

| S.No | Ingredients | mg/ml |
|---|---|---|
| 1 | Nitrofurantoin | 10.000 |
| 2 | Carboxy methylcellulose sodium | 12.000 |
| 3 | Magnesium aluminum silicate | 9.000 |
| 4 | Methyl paraben | 1.200 |
| 5 | Propyl paraben | 0.200 |
| 6 | Glycerin | 140.000 |
| 7 | Sorbitol solution | 140.000 |
| 8 | Sodium citrate dihydrate | 4.000 |
| 9 | Citric acid (anhydrous) | 1.360 |
| 10 | Primary taste modifier | 9.000 |
| 11 | Banana flavor | 0.050 |
| 12 | Rasberry flavor | 0.100 |

The aqueous pharmaceutical composition of this example consists of 50 mg/5 ml of nitrofurantoin and has a pH of 5.1 to 5.7. The lead content in the above formulation is 3.07 PPM.

### Example 6

According to one embodiment, the aqueous pharmaceutical composition of nitrofurantoin is as follow:

| S.No | Ingredients | mg/ml |
|---|---|---|
| 1 | Nitrofurantom monohydrate | 10.000 |
| 2 | Carboxymethylcellulose sodium | 14.000 |
| 3 | Magnesium aluminum silicate | 8.000 |
| 4 | Methyl paraben | 1.200 |
| 5 | Propyl paraben | 0.200 |
| 6 | Glycerin | 140.000 |
| 7 | Sorbitol solution | 140.000 |
| 8 | Sodium citrate dihydrate | 4.000 |
| 9 | Citric acid (anhydrous) | 1.360 |
| 10 | Primary taste modifier | 9.000 |
| 11 | Banana flavor | 0.050 |
| 12 | Rasberry flavor | 0.100 |

### METHOD OF PREPARATION

Heat Purified water to a temperature between 55° C-60 °C (target 57 °C) and add the binding agent and suspending agent after the water reaches a temperature of 57 °C, while mixing and homogenising for over a period 40 to 60 minutes. Add purified water to bring up the weight (56.82% to final target w eight) and continue agitation for at least 75 minutes until a smooth and uniform product is obtained. Add and disperse the preservative to Purified water with mixing and homogenisation to form a dispersion. Transfer this dispersion to the above mixture and set the temperature to 20-30 °C. Add vehicle and sweetening agent to the above dispersion and mix for at least 90 minutes until the dispersion is complete and then add buffering agent and keep mixing for at least 15 minutes. Now add Nitrofurantoin to this dispersion and then transfer to above dispersion and mix for at least 15 minutes until a smooth suspension is obtained.

### Example 7

The aqueous pharmaceutical composition as claimed herein is made by heat purified water to a temperature between 55 °C-60 °C (target 57 °C) and adding carboxymethylcellulose and magnesium aluminium silicate after the water reaches a temperature of 57 °C, while mixing and homogenising for over a period 40 to 60 minutes. Add purified water to bring up the weight (56.82% to final target weight) and continue agitation for at least 75 minutes until a smooth and uniform product is obtained. Add and disperse the methyl paraben and propyl paraben, one at a time, to the purified water with mixing and homogenisation to form a dispersion. Transfer this dispersion to the above mixture and set the temperature to 20-30 °C. Add glycerin and sorbitol solution to the above dispersion and mix for at least 90 minutes until the dispersion is complete and then add sodium citrate and citric acid, and keep mixing for at least 15 minutes. Now add Nitrofurantoin to this dispersion and then transfer to above dispersion and mix for at least 15 minutes until a smooth suspension is obtained.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1-6. (canceled)

7. A formulation comprising a uniform composition comprising:
a) nitrofurantoin or its pharmaceutically acceptable salt or a polymorph thereof in an amount of 5 mg/mL or 10 mg/mL;
b) a suspending agent comprising magnesium aluminum silicate and carboxymethylcellulose sodium; wherein the magnesium aluminum silicate is in an amount of about 4 mg/mL and carboxymethylcellulose sodium is in an amount of about 20 mg/mL when the concentration of nitrofurantoin or its pharmaceutically acceptable salt or a polymorph thereof is about 5 mg/mL, and magnesium aluminum silicate is in an amount from about 8 mg/mL to about 9 mg/mL and carboxymethylcellulose sodium is in an amount from about 12 mg/mL to about 14 mg/mL when the concentration of nitrofurantoin or its pharmaceutically acceptable salt or a polymorph thereof is about 10 mg/mL;
c) glycerin and/or sorbitol;
d) methyl paraben; and
e) propyl paraben;
wherein the amount of the methyl paraben is at least 6 times that of the propyl paraben;
wherein pH of the formulation is in a range of 4.5 to 6.5; and
wherein the formulation is an aqueous pharmaceutical suspension suitable for oral administration.

8. The formulation of claim 7, wherein lead content is between 3.07 ppm to 5 ppm.

9. The formulation of claim 7, wherein pH of the formulation is 5.1 to 5.7.

10. The formulation of claim 7, further comprises a taste masking agent in an amount of 0.01 wt, to 2.0 wt. %.

11. The formulation of claim 7, wherein the formulation has lead content between 3.07 ppm to 4 ppm.

12. The formulation of claim 7, wherein the formulation comprises glycerin and sorbitol.

13. The formulation of claim 13, wherein the formulation comprises an equal amount of glycerin and sorbitol.

14. The formulation of claim 7, further comprising a buffering agent.

15. An aqueous pharmaceutical suspension for oral administration comprising a formulation comprising nitrofurantoin or its pharmaceutically acceptable salt or polymorph thereof in an amount of about 5 mg/mL or about 10 mg/mL, a suspending agent consisting of magnesium aluminum silicate in an amount of about 4 mg/mL and carboxymethylcellulose sodium in an amount of about 20 mg/mL when the concentration of nitrofurantoin or its pharmaceutically acceptable salt or polymorph thereof is about 5 mg/mL, and magnesium aluminum silicate in an amount from about 8 mg/mL to about 9 mg/mL and carboxymethylcellulose sodium in an amount from about 12 mg/mL to about 14 mg/mL when the concentration of nitrofurantoin or its pharmaceutically acceptable salt or polymorph thereof is about 10 mg/mL, and lead; wherein the aqueous pharmaceutical suspension contains less than 5 ppm of lead as an impurity.

16. The aqueous pharmaceutical suspension of claim 15, wherein the amount of lead is less than 3.5 ppm.

17. The aqueous pharmaceutical suspension of claim 15, wherein the nitrofurantoin or its pharmaceutically acceptable salt or the polymorph is in an amount of about 5 mg/ml to 10 mg/ml.

18. The aqueous pharmaceutical suspension of claim 15, wherein pH of the formulation is in a range of 4.5 to 6.5.

19. The aqueous pharmaceutical suspension of claim 15, wherein the formulation is a uniform product.

20. The aqueous pharmaceutical suspension of claim 15, wherein the formulation further comprises methyl paraben and propyl paraben, wherein the amount of methyl paraben is at least 6 times more than propyl paraben.

21. The aqueous pharmaceutical suspension of claim 15, wherein the nitrofurantoin or its pharmaceutically acceptable salt or the polymorph is in an amount of about 5 mg/ml, and the amount of lead is less than 3.5 ppm.

22. The aqueous pharmaceutical suspension of claim 15, wherein the nitrofurantoin or its pharmaceutically acceptable salt or the polymorph is in an amount of about 10 mg/ml, and the amount of lead is less than 3.5 ppm.

23. An aqueous pharmaceutical suspension for oral administration comprising a formulation comprising nitrofurantoin or its pharmaceutically acceptable salt or polymorph in an amount of about 5 mg/mL or about 10 mg/mL, a suspending agent consisting of magnesium aluminum silicate in an amount of about 4 mg/mL and carboxymethylcellulose sodium in an amount of about 20 mg/mL when the concentration of nitrofurantoin or its pharmaceutically acceptable salt or polymorph thereof is about 5 mg/mL, and magnesium aluminum silicate in an amount from about 8 mg/mL to about 9 mg/mL and carboxymethylcellulose sodium in an amount from about 12 mg/mL to about 14 mg/mL when the concentration of nitrofurantoin or its pharmaceutically acceptable salt or polymorph thereof is about 10 mg/mL;
wherein the aqueous pharmaceutical suspension is a uniform product; and
wherein the aqueous pharmaceutical suspension contains less than 3.5 ppm lead as an impurity.
